SEQUENCE LISTING (3 sequences)

SEQ ID NO: 1 - protein, 764 aa, Bacillus anthracis
MKKRKVLIPLMALSTILVSSTGNLEVIQAEVKQENRLLNESESSSQGLLGYYFSDLNFQAPMVVTSSTTGDLSIPSSELENIPSENQYFQSAIWSGFIKVKKSDEYTFATSADNHVTMWVDDQEVINKASNSNKIRLEKGRLYQIKIQYQRENPTEKGLDFKLYWTDSQNKKEVISSDNLQLPELKQKSSNSRKKRSTSAGPTVPDRDNDGIPDSLEVEGYTVDVKNKRTFLSPWISNIHEKKGLTKYKSSPEKWSTASDPYSDFEKVTGRIDKNVSPEARHPLVAAYPIVHVDMENIILSKNEDQSTQNTDSQTRTISKNTSTSRTHTSEVHGNAEVHASFFDIGGSVSAGFSNSNSSTVAIDHSLSLAGERTWAETMGLNTADTARLNANIRYVNTGTAPIYNVLPTTSLVLGKNQTLATIKAKENQLSQILAPNNYYPSKNLAPIALNAQDDFSSTPITMNYNQFLELEKTKQLRLDTDQVYGNIATYNFENGRVRVDTGSNWSEVLPQIQETTARIIFNGKDLNLVERRIAAVNPSDPLETTKPDMTLKEALKIAFGFNEPNGNLQYQGKDITEFDFNFDQQTSQNIKNQLAELNATNIYTVLDKIKLNAKMNILIRDKRFHYDRNNIAVGADESVVKEAHREVINSSTEGLLLNIDKDIRKILSGYIVEIEDTEGLKEVINDRYDMLNISSLRQDGKTFIDFKKYNDKLPLYISNPNYKVNVYAVTKENTIINPSENGDTSTNGIKKILIFSKKGYEIG

SEQ ID NO: 2 - protein, 4 aa, Artificial Sequence
  Description of Artificial Sequence Synthetic peptide
SSSR

SEQ ID NO: 3 - protein, 40 aa, Artificial Sequence
  Description of Artificial Sequence Synthetic peptide
GGGGSGGGGSGGGGSGGGGSGGGGSGGGGSGGGGSGGGGS

(19) United States
(12) Patent Application Publication
(10) Pub. No.: US 2009/0092652 A1
(43) Pub. Date: Apr. 9, 2009

(54) Method for the inhibition of angiogenesis or cancer using protective antigen related molecules

(75) Inventors: Kenneth Christensen, Clemson, SC (US); Michael S. Rogers, Needham, MA (US); Robert J. D'Amato, Lexington, MA (US)

Correspondence Address: DAVID S. RESNICK, NIXON PEABODY LLP, 100 SUMMER STREET, BOSTON, MA, 02110-2131, US

(73) Assignees: Children's Medical Center Corporation, Boston, MA (US); President and Fellows of Harvard College, Cambridge, MA (US)

(21) Appl. No.: 11/660,254

(22) PCT Filed: Aug. 10, 2005
(86) PCT No.: PCT/US2005/028296

§ 371 Date: Oct. 20, 2008

Related U.S. Application Data
(60) Provisional application No. 60/603,239, filed on Aug. 20, 2004.

(51) Int. Cl. A61K 9/70; A61K 39/07; A61P 35/00

(52) U.S. Cl. 424/423; 424/246.1

(57) ABSTRACT

The present invention is based on the discovery that protective antigen related molecules (PARMs) without anthrax lethal factor have antiangiogenic or anticancer properties. The invention is directed to a method of inhibiting an angiogenic disease/disorder or cancer. Additionally, the invention can be applied to those at risk for developing cancer or an angiogenic disease/disorder comprising administering to a mammal an angiogenesis-inhibiting or cancer inhibiting amount of an PARM (including analogs, or derivative thereof having angiogenesis-inhibiting or anticancer activity, consisting of PA, PA fragment, analog, or derivative that is administered in a composition substantially free of anthrax lethal factor or other toxins).

### CROSS-REFERENCE TO RELATED APPLICATIONS

This application claims the benefit under 35 U.S.C. § 119(e) of U.S. provisional Patent Application No. 60/603,239, filed Aug. 20, 2004.

### FIELD OF INVENTION

The present invention relates to a method for treatment of cancer or diseases/disorders involving angiogenesis.

### BACKGROUND OF THE INVENTION

Angiogenesis is a process of tissue vascularization that involves the growth of new blood vessels into a tissue, and is also referred to as neo-vascularization. Blood vessels are the means by which oxygen and nutrients are supplied to living tissues and waste products are removed from living tissue. When appropriate, angiogenesis is a critical biological process. For example, angiogenesis is essential in reproduction, development and wound repair. Conversely, inappropriate angiogenesis can have severe negative consequences. For example, it is only after solid tumors are vascularized as a result of angiogenesis that the tumors have a sufficient supply of oxygen and nutrients that permit it to grow rapidly and metastasize.

One example of a disease mediated by angiogenesis is ocular neovascular disease. This disease is characterized by invasion of new blood vessels into the structures of the eye such as the retina or cornea. It is the most common cause of blindness and is involved in approximately twenty eye diseases. In age-related macular degeneration, the associated visual problems are caused by an ingrowth of chorioidal capillaries through defects in Bruch's membrane with proliferation of fibrovascular tissue beneath the retinal pigment epithelium. Angiogenic damage is also associated with diabetic retinopathy, retinopathy of prematurity, corneal graft rejection, neovascular glaucoma and retrolental fibroplasia. Other diseases associated with corneal neovascularization include, but are not limited to, epidemic keratoconjunctivitis, Vitamin A deficiency, contact lens overwear, atopic keratitis, superior limbic keratitis, pterygium keratitis sicca, sjogrens, acne rosacea, phylectenulosis, syphilis, Mycobacteria infections, lipid degeneration, chemical burns, bacterial ulcers, fungal ulcers, Herpes simplex infections, Herpes zoster infections, protozoan infections, Kaposi's sarcoma, Mooren's ulcer, Terrien's marginal degeneration, mariginal keratolysis, rheumatoid arthritis, systemic lupus, polyarteritis, trauma, Wegener's sarcoidosis, scleritis, Stevens-Johnson disease, pemphigoid, radial keratotomy, and corneal graph rejection.

Diseases associated with retinal/choroidal neovascularization include, but are not limited to, diabetic retinopathy, macular degeneration, sickle cell anemia, sarcoid, syphilis, pseudoxanthoma elasticum, Paget's disease, vein occlusion, artery occlusion, carotid obstructive disease, chronic uveitis/vitritis, mycobacterial infections, Lyme's disease, systemic lupus erythematosis, retinopathy of prematurity, Eales' disease, Behcet's disease, infections causing a retinitis or choroiditis, presumed ocular histoplasmosis, Best's disease, myopia, optic pits, Stargardt's disease, pars planitis, chronic retinal detachment, hyperviscosity syndromes, toxoplasmosis, trauma and post-laser complications. Other diseases include, but are not limited to, diseases associated with rubeosis (neovasculariation of the angle) and diseases caused by the abnormal proliferation of fibrovascular or fibrous tissue including all forms of proliferative vitreoretinopathy.

Another disease in which angiogenesis is believed to be involved is rheumatoid arthritis. The blood vessels in the synovial lining of the joints undergo angiogenesis. In addition to forming new vascular networks, the endothelial cells release factors and reactive oxygen species that lead to pannus growth and cartilage destruction. The factors involved in angiogenesis may actively contribute to, and help maintain, the chronically inflamed state of rheumatoid arthritis.

Factors associated with angiogenesis may also have a role in osteoarthritis. The activation of the chondrocytes by angiogenic-related factors contributes to the destruction of the joint. At a later stage, the angiogenic factors would promote new bone formation. Therapeutic intervention that prevents the bone destruction could halt the progress of the disease and provide relief for persons suffering with arthritis.

Chronic inflammation may also involve pathological angiogenesis. Such disease states as ulcerative colitis and Crohn's disease show histological changes with the ingrowth of new blood vessels into the inflamed tissues. Bartonellosis, a bacterial infection found in South America, can result in a chronic stage that is characterized by proliferation of vascular endothelial cells. Another pathological role associated with angiogenesis is found in atherosclerosis. The plaques formed within the lumen of blood vessels have been shown to have angiogenic stimulatory activity. Inhibitors of angiogenesis could be useful to prevent atherosclerosis progression or plaque restenosis after angioplasty.

One of the most frequent angiogenic diseases of childhood is the hemangioma. In most cases, the tumors are benign and regress without intervention. In more severe cases, the tumors progress to large cavernous and infiltrative forms and create clinical complications. Systemic forms of hemangiomas, the hemangiomatoses, have a high mortality rate. Therapy-resistant hemangiomas exist that cannot be treated with therapeutics currently in use.

Angiogenesis is also responsible for damage found in hereditary diseases such as Osler-Weber-Rendu disease, or hereditary hemorrhagic telangiectasia. This is an inherited disease characterized by multiple small angiomas, tumors of blood or lymph vessels. The angiomas are found in the skin and mucous membranes, often accompanied by epistaxis (nosebleeds) or gastrointestinal bleeding and sometimes with pulmonary or hepatic arteriovenous fistula. In addition, dysregulated angiogenesis is responsible for Klippel-Trenaunay syndrome which is characterized by malformations of capillary, venous, and lymphatic vessels; and by bony and soft tissue hypertrophy.

Angiogenesis is prominent in solid tumor formation and metastasis. Angiogenic factors have been found associated with several solid tumors such as rhabdomyosarcomas, retinoblastoma, Ewing sarcoma, neuroblastoma, and osteosarcoma. A tumor cannot expand without a blood supply to provide nutrients and remove cellular wastes. Tumors in which angiogenesis is important include solid tumors (prostate, breast, lung, colon, uterine, skin, ovarian . . . ) and benign tumors such as acoustic neuroma, neurofibroma, trachoma and pyogenic granulomas. Prevention of angiogenesis could halt the growth of these tumors and the resultant damage to the animal due to the presence of the tumor.

It should be noted that angiogenesis has been associated with blood-born tumors such as leukemias, any of various acute or chronic neoplastic diseases of the bone marrow in which unrestrained proliferation of white blood cells occurs, usually accompanied by anemia, impaired blood clotting, and enlargement of the lymph nodes, liver, and spleen. It is believed that angiogenesis plays a role in the abnormalities in the bone marrow that give rise to leukemia-like tumors and other diseases such as multiple myeloma and lymphoma.

Angiogenesis is important in two stages of tumor metastasis. The first stage where angiogenesis stimulation is important is in the vascularization of the tumor which allows tumor cells to enter the blood stream and to circulate throughout the body. After the tumor cells have left the primary site, and have settled into the secondary, metastasis site, angiogenesis must occur before the new tumor can grow and expand. Therefore, prevention of angiogenesis could lead to the prevention of metastasis of tumors and possibly contain the neoplastic growth at the primary site.

Knowledge of the role of angiogenesis in the maintenance and metastasis of tumors has led to a prognostic indicator for breast cancer. The amount of neovascularization found in the primary tumor was determined by counting the microvessel density in the area of the most intense neovascularization in invasive breast carcinoma. A high level of microvessel density was found to correlate with tumor recurrence. Control of angiogenesis by therapeutic means could possibly lead to cessation of the recurrence of the tumors.

Angiogenesis is also involved in normal physiological processes such as reproduction and wound healing. Angiogenesis is an important step in ovulation, endometrial proliferation and also in implantation of the blastula after fertilization. Prevention of angiogenesis could be used to induce amenorrhea, to block ovulation, to prevent implantation by the blastula and to inhibit endometriosis. Angiogenesis is also involved in other normal physiological processes such as fat accumulation and expansion. Thus angiogenesis inhibition is useful to treat obesity.

In wound healing, excessive repair or fibroplasia can be a detrimental side effect of surgical procedures and may be caused or exacerbated by angiogenesis. Adhesions are a frequent complication of surgery and lead to problems such as small bowel obstruction.

In a recent review by Folkman, it was estimated that more than one-third of all women between the ages of 40 and 50 have in-situ tumors in their breasts. Such tumors lie dormant in the body and rarely, if ever, are diagnosed as breast cancer. It is believed that a similar phenomenon exists in men in regards to prostate cancer. In light of such data, cancer might be defined as having two distinct phases: (1) Acquisition of mutations which transform normal cells into cancerous cells, and the formation of in-situ tumors; and (2) A switch to an angiogenic phenotype, whereby the in-situ tumor is supplied with new blood vessels, supporting rapid tumor growth and metastasis (Nature, Vol. 427, Feb. 26, 2004, p. 787). Therapeutic compounds that are able to prevent the switch to an angiogenic phenotype (i.e. from an in-situ tumor to a rapidly growing tumor), are needed to prevent the onset of tumor growth. Angiogenesis inhibitors have shown promise in animal studies and clinical trials are currently underway (Kerbel et al. Nature Reviews, Vol. 2, pp. 727-739). However, new compounds that inhibit angiogenesis are needed.

Anthrax protective antigen (PA) is an 83 kDa protein derived from Bacillus anthracis. Bacillus anthracis, is a gram-positive, spore forming, rod-shaped bacterium that carries the well known Anthrax toxin. The toxin is formed by three proteins; protective antigen (PA), lethal factor (LF), and edema factor (EF). Individually, none of the three toxin associated proteins are toxic. However, a mixture of PA and LF (called lethal toxin; LeTx) is known to cause lethal shock in animals. PA, EF, and LF can form toxic complexes either in solution or on the cell surface. When PA binds to a cell surface receptor and is activated by a furin protease, assembly of the three toxin proteins occurs (Bradley et al., Nature 414:225-29, 2001). Cellular proteases from the furin family cleave PA into two fragments: PA₂₀ (20 kDa, N-terminal portion) and PA₆₃ (C-terminal portion). While PA₆₃ remains associated with the PA cellular receptor, PA₂₀ dissociates. Receptor bound PA₆₃ then self-oligomerizes to form a ring-shaped pore (Milene et al., J. Bil. Chem. 269:20607-20612, 1994). EF and LF then binds to the PA₆₃ subunits (Cunningham et al., Proc. Natl. Acad. Sci. USA 99:6603-6606, 2002) forming complexes that enter the cell by endocytosis. Once inside the cell PA forms a pore in the endosome and releases LF and EF into the cytosol where LF and EF are active. PA is the most immunogenic protein of the toxin and immunization against PA is protective against anthrax toxicity (Friedlander et al., Curr. Top. Microbiol. Immunol. 271:33-60, 2002). Thus, PA with amino acid sequences identical to the natural forms have been produced by chemical synthesis as well as by recombinant technology and used for vaccine development. PA alone has not been previously demonstrated to inhibit either tumor growth or angiogenisis. As PA is endocytosed and forms transmembrane pores, PA has been used as a delivery vehicle for other proteins in the treatment of cancer, e.g., PA20-toxin fusion proteins (U.S. Pat. No. 5,677,274) or anthrax lethal factor. Anthrax lethal factor (LF) is a protease which cleaves MEKs (Map Kinase Kinase). Given the importance of MEK signaling in tumorigenesis, the effects of lethal factor (LF) on tumor growth have been studied by delivering LF into the cell via treatment with whole anthrax toxin (LeTx) (a mixture of protective antigen (PA) and lethal factor (LF)). LeTx was found to be effective at inhibiting growth of human melanoma xenograft tumors in athymic nude mice (Koo et. al., Proc. Natl. Acad. Sci. 99(5): 3052-3057 2002). In these experiments, LF was found to be the component responsible for inhibition of tumor growth. In fact, PA alone was used as a control and the authors concluded that there is no inhibition of tumor growth by PA. In addition, in a separate study, LeTx was found to decrease tumor neovascularization and to effectively inhibit growth of ras-transformed cells implanted in athymic nude mice (U.S. Patent Application 20040136975). However, here too, the effects of LeTx were attributed to anthrax lethal factor.

Protective antigen related molecules (PARM) refers to compounds which are either PA, analogs of PA, fragments of PA (contiguous or noncontiguous) or synthetic peptides based partly on PA sequence.

### SUMMARY

The present invention is based on the discovery that protective antigen related molecules (PARMs) without anthrax lethal factor have antiangiogenic or anticancer properties. The invention is directed to a method of inhibiting an angiogenic disease/disorder or cancer. Additionally, the invention can be applied to those at risk for developing cancer or an angiogenic disease/disorder comprising administering to a mammal an angiogenesis-inhibiting or cancer inhibiting amount of an PARM (including analogs, or derivative thereof having angiogenesis-inhibiting or anticancer activity, consisting of PA, PA fragment, analog, or derivative that is administered in a composition substantially free of anthrax lethal factor or other toxins).

As used herein, “substantially free of anthrax lethal factor or other toxins” is meant to indicate that the lethal factor or other toxins (e.g. exotoxin, diphtheria toxin, Shiga toxin, or ricin) can be present in an incidental amount. In other words, the material is not intentionally added to an indicated composition, but may be present at a minor or inconsequential levels, for example, because it was carried over as an impurity as part of an intended composition component.

In one embodiment of the present invention, PARM comprises full length PA, amino acids 1-764 of SEQ ID NO.: 1, preferably, amino acids 30-764 of SEQ ID NO: 1. Amino acids 1-29 of SEQ ID NO: 1 encode the PA signal sequence. Alternatively, PARM may be an angiogenesis-inhibiting fragment, analog, or derivative of SEQ ID NO. 1. In one preferred embodiment, PA fragments, analogs or derivatives thereof, which are derived from SEQ ID NO: 1, are linked together by peptide or other linkers or by using standard coupling chemistries. In one embodiment, PARM is a peptide or peptides selected from the groups consisting of amino acids 365-384 of SEQ ID NO.: 1; and/or amino acids 708-721 of SEQ ID NO.: 1; and/or amino acids 676-694 of SEQ ID NO.: 1; and/or amino acids 732-751 of SEQ ID NO.: 1; and/or amino acids 595-764 of SEQ ID NO.: 1.

In yet another embodiment, PARM comprises a fragment of PA having at least 50% identity compared to a fragment of PA from which the peptide was derived, wherein the fragment is derived from SEQ ID NO. 1.

In still another embodiment, PARM is a mutant PA that is not cleaved by the protease furin, such as described in Klimpel et al. Proc. Natl. Acad. Sci. USA 89: 10277-10281, 1992. In one preferred embodiment, the mutant that is not cleaved by furin, herein referred to as SSSR, has the sequence Ser-Ser-Ser-Arg inserted in place of the sequence of Arg-Lys-Lys-Arg found at amino acid residues 193-196 of SEQ ID NO:1.

Furthermore, the present invention is directed to method of inhibiting angiogenesis and/or cancer in a tissue of a mammal having an angiogenic disease and/or cancer.

In another embodiment of the present invention, the methods are directed to the treatment of a solid tumor or solid tumor metastasis.

In another embodiment of the present invention, the methods are directed to the treatment of a blood borne or bone marrow derived tumors such as leukemia, multiple myleloma or lymphoma.

In yet another embodiment, the methods are directed to the treatment of retinal tissue and said disease or disorder is retinopathy, diabetic retinopathy, or macular degeneration.

In yet another embodiment, the methods of the present invention are directed toward treatment of atherosclerosis or a tissue at risk of restenosis, wherein the tissue is at the site of coronary angioplasty.

In another embodiment of the present invention, the methods are directed toward inhibiting angiogenesis in a tissue of a mammal, wherein said tissue is inflamed and said disease or disorder is arthritis (rheumatoid or osteo-arthritis).

The methods of the present invention can be used either alone, or in conjunction with other treatment methods known to those of skill in the art. Such methods may include, but are not limited to, chemotherapy, radiation therapy, or other known angiogenesis inhibitors.

In yet another embodiment of the present invention, said administering comprised intravenous, transdermal, intrasynovial, intramuscular, intraocular/periocular or oral administration. Alternatively, administration of PARM may comprise administering a gene therapy vector that constitutively or inducibly expresses PA, PA derivative, or fragments thereof.

The methods of the present invention allow for the administration of PARM either prophylactically or therapeutically.

Finally, the methods of the present invention are directed toward inhibiting cancer or an angiogenic disease or disorder in a mammal at risk for developing cancer or an angiogenic disease or disorder. The risk can be determined genetically. Alternatively, the risk can be determined by measuring levels of cancer marker proteins in the biological fluids (i.e. blood, urine) of a patient. Marker proteins include, for example, calcitonin, PSA, thymosin β-15, thymosin β-16, and matrix metalloproteinases (MMPs).

### BRIEF DESCRIPTION OF FIGURES

FIG. 1 shows a graph of inhibition of neovascularization in a standard angiogenesis assay by SSSR, a mutant version of PA that can not be cleaved by furin to promote the formation of PA oligomers and internalization.

FIG. 2 shows a graph of inhibition of lewis-lung cell carcinoma tumor growth by SSSR in C57BL/6J mice.

FIG. 3 shows a graph of inhibition of neovascualrization in a standard angiogenenesis assay by SSSR versus wild type PA. While both SSSR and wild type PA inhibit angiogenesis, greater activity was seen with SSSR.

### DETAILED DESCRIPTION

### Definitions

PARM (protective antigen related molecules) refers to compounds which are either native PA, analogs of PA, fragments of PA (contiguous or noncontiguous) or synthetic peptides based partly on PA sequence.

Amino Acid Residue: An amino acid formed upon chemical digestion (hydrolysis) of a polypeptide at its peptide linkages. The amino acid residues described herein are preferably in the “L” isomeric form. However, residues in the “D” isomeric form can be substituted for any L-amino acid residue, as long as the desired functional property is retained by the polypeptide. NH₂ refers to the free amino group present at the amino terminus of a polypeptide. COOH refers to the free carboxy group present at the carboxy terminus of a polypeptide. In keeping with standard polypeptide nomenclature (described in J. Biol. Chem., 243:3552-59 (1969) and adopted at 37 CFR .sctn. 1.822 (b) (2)), abbreviations for amino acid residues are shown in the following Table of Correspondence:

TABLE OF CORRESPONDENCE
SYMBOL

| 1-Letter | 3-Letter | AMINO ACID |
|---|---|---|
| Y | Tyr | tyrosine |
| G | Gly | glycine |
| F | Phe | phenylalanine |
| M | Met | methionine |
| A | Ala | alanine |
| S | Ser | serine |
| I | Ile | isoleucine |
| L | Leu | leucine |
| T | Thr | threonine |
| V | Val | valine |
| P | Pro | proline |
| K | Lys | lysine |
| H | His | histidine |
| Q | Gln | glutamine |
| E | Glu | glutamic acid |
| Z | Glx | Glu and/or Gln |
| W | Trp | tryptophan |
| R | Arg | arginine |
| D | Asp | aspartic acid |
| N | Asn | asparagine |
| B | Asx | Asn and/or Asp |
| C | Cys | cysteine |
| X | Xaa | unknown/other |

It should be noted that all amino acid residue sequences are represented herein by formulae whose left and right orientation is in the conventional direction of amino-terminus to carboxy-terminus. Furthermore, it should be noted that a dash at the beginning or end of an amino acid residue sequence indicates a peptide bond to a further sequence of one or more amino acid residues.

Polypeptide: refers to a linear series of amino acid residues connected to one another by peptide bonds between the alpha-amino group and carboxy group of contiguous amino acid residues.

Peptide: as used herein refers to a linear series of no more than about 50 amino acid residues connected one to the other as in a polypeptide.

Cyclic peptide: refers to a compound having a heteroatom ring structure that includes several amide bonds as in a typical peptide. The cyclic peptide can be a “head to tail” cyclized linear polypeptide in which a linear peptide's n-terminus has formed an amide bond with the terminal carboxylate of the linear peptide, or it can contain a ring structure in which the polymer is homodetic or heterodetic and comprises amide bonds and/or other bonds to close the ring, such as disulfide bridges, thioesters, thioamides, guanidino, and the like linkages.

Protein: refers to a linear series of greater than 50 amino acid residues connected one to the other as in a polypeptide.

Fusion protein: refers to a polypeptide containing at least two different polypeptide domains operatively linked by a typical peptide bond (“fused”), where the two domains correspond to peptides no found fused in nature.

Synthetic peptide: refers to a chemically produced chain of amino acid residues linked together by peptide bonds that is free of naturally occurring proteins and fragments thereof.

As it is used herein the term “PARM” is intended to include native PA, PA homologues, derivatives, fragments thereof, analogs and mimetics (whether or not the later terms are listed after an occurrence of “PARM”), when administered, the PARM is free of and not associated with anthrax lethal factor or other toxins. A “PARM mimetic” is an agent, generally a peptide or polypeptide molecule, that recognizes a PA receptor (Bradley et al., Nature 414:225-29, 2001; Scobie et al., 2003 Proc. Natl. Acad. Sci. USA 100:5170-74). PA receptors include, but are not limited to TEM8 (Bradely et al. Nature 414, 225-229, 2001) and CMG2 (Wigelsworth, et al. J Biol Chem 279, 23349-23356, 2004). PARM is also intended to include peptide molecules that are linked via a peptide or other linker. For example, full length PA, is an 83 kDA protein (SEQ ID NO: 1) that has 4 domains. The PA for use in the invention can consist of domains 2 and 4 linked by a peptide or other linker, or can consist of any combination of domains, fragments, or derivatives thereof, from full length PA. The domains and crystal structure of PA are described in Collier et al., Annu. Rev. Cell Biol. 19: 45-70, 2003, which is herein incorporated by reference in its entirety. The crystal structure of PA in complex with one of its receptors is also known (Eugenio Santelli, Laurie A. Bankston, Stephen H. Leppla, Robert C. Liddington, 2004, Nature, 430(7002):905-908). Using structural information elucidated by crystallography or other high resolution methods, amino acid sequences in PA that are involved in receptor binding can be identified and these sequences used to generate molecules that will inhibit angiogenesis or cancer. Examples of such sequences are provided herein.

### General Considerations

The present invention relates generally to a method of inhibiting angiogenesis and/or cancer in a mammal having an angiogenic disease or cancer. The method of the present invention comprises the administration of an effective amount of PARM having antiangiogenic and/or anticancer activity to a mammal. Although the compounds disclosed herein have both antiangiogenic and anticancer properties, we conceive that these effects may be independent and thus PARMs may have either antiangiogenic or anticancer activity or both.

Angiogenesis plays a role in a variety of disease processes. By inhibiting angiogenesis, one can intervene in the disease, ameliorate the symptoms, and in some cases cure the disease. Where the growth of new blood vessels is the cause of, or contributes to, the pathology associated with a disease, inhibition of angiogenesis will reduce the deleterious effects of the disease. Examples include rheumatoid arthritis, obesity, diabetic retinopathy, inflammatory diseases, restenosis, and the like. Where the growth of new blood vessels is required to support growth of a deleterious tissue, inhibition of angiogenesis will reduce the blood supply to the tissue and thereby contribute to reduction in tissue mass based on blood supply requirements. Examples include growth of tumors where neovascularization is a continual requirement in order that the tumor grows beyond a few millimeters in thickness, and for the establishment of solid tumor metastases.

The invention provides for a method for the inhibition of angiogenesis in a tissue, and thereby inhibiting events in the tissue which depend upon angiogenesis. Generally, the method comprises administering to the tissue a composition comprising an angiogenesis-inhibiting amount of PARM. In one embodiment of the present invention, PARM comprises full length PA, herein described as SEQ ID NO.: 1, preferably amino acids 30-764 of SEQ ID NO: 1. Alternatively, PARM may be an angiogenesis-inhibiting fragment, analog, or derivative of SEQ ID NO.: 1. PARMs useful in the treatment of angiogenic diseases as described in the present invention will inhibit angiogenesis in the corneal neovascularization assay (Gimbrone, M A. et al. (1974) J Natl Canc Inst. 52:413-427; Kenyon, B M. et al. (1996) Invest Opthalmol V is Sci 37:1625-1632; Kenyon, B M. et al. (1997) Exp Eye Res 64:971-97; Proia, A D. et al. (1993) Exp Eye Res 57:693-698) by at least 25%, more preferably, by at least 50%. In one preferred embodiment, PARM comprises amino acids 365-384 of SEQ ID NO.: 1, and/or amino acids 708-721 of SEQ ID NO.: 1, and/or amino acids 676-694 of SEQ ID NO.: 1, and/or amino acids 732-751 of SEQ ID NO.: 1. Peptides, analogs, or derivatives thereof derived from SEQ ID NO: 1 can be linked together by peptide or other linkers or by using standard coupling chemistries. Such fragments can be at least 8, 10, 20, 30, 40, 50, 75, 100, or 150 amino acids in length.

In one embodiment, PARM comprises a derivative of SEQ ID NO.:1 having at least 50% identity compared to a fragment of PA from which the derivative was derived.

In another embodiment, PARM is a mutant PA that is not cleaved by furin, for example, SSSR as described in Klimpel et al., Proc Natl Acad Sci USA. November 1; 89(21):10277-10281, 1992, which is herein incorporated by reference.

### Angiogenesis Screening Assays

Examples of well described angiogenesis screening assays that may be initially used to test the antiangiogenic activity of PARM include, but are not limited to, in vitro endothelial cell assays, rat aortic ring angiogenesis assays, cornea micropocket assays (corneal neovascularization assays), and chick embryo chorioallantoic membrane assays (Erwin, A. et al. (2001) Seminars in Oncology 28(6):570-576).

Some example in vitro endothelial cell assays include methods for monitoring endothelial cell proliferation, cell migration, or tube formation. Cell proliferation assays may use cell counting, BRdU incorporation, thymidine incorporation, or staining techniques (Montesano, R. (1992) Eur J Clin Invest 22:504-515; Montesano, R. (1986) Proc Natl. Acad. Sci. USA 83:7297-7301; Holmgren L. et al. (1995) Nature Med 1: 149-153).

In the cell migration assays endothelial cells are plated on matrigel and migration monitored upon addition of a chemoattractant (Homgren, L. et al. (1995) Nature Med 1:149-153; Albini, A. et al. (1987) Cancer Res. 47:3239-3245; Hu, G. et al. (1994) Proc Natl Acad Sci USA 6:12096-12100; Alessandri, G. et al. (1983) Cancer Res. 43:1790-1797.)

The endothelial tube formation assays monitor vessel formation (Kohn, E C. et al. (1995) Proc Natl Acad Sci USA 92:1307-1311; Schnaper, H W. et al. (1995) Cell Physiol 165:107-118).

Rat aortic ring assays have been used successfully for the screening of angiogenesis drugs (Zhu, W H. et al. (2000) Lab Invest 80:545-555; Kruger, E A. et al. (2000) Invasion Metastas 18:209-218; Kruger, E A. et al. (2000) Biochem Biophys Res Commun 268:183-191; Bauer, K S. et al. (1998) Biochem Pharmacol 55:1827-1834; Bauer, K S. et al. (2000) J Pharmacol Exp Ther 292:31-37; Berger, A C. et al. (2000) Microvasc Res 60:70-80.). Briefly, the assay is an ex vivo model of explant rat aortic ring cultures in a three dimensional matrix. One can visually observe either the presence or absence of microvessel outgrowths. The human saphenous angiogenesis assay, another ex vivo assay, may also be used (Kruger, E A. et al. (2000) Biochem Biophys Res Commun 268:183-191).

Another common screening assay is the cornea micropocket assay (Gimbrone, M A. et al. (1974) J Natl Canc Inst. 52:413-427; Kenyon, B M. et al. (1996) Invest Opthalmol V is Sci 37:1625-1632; Kenyon, B M. et al. (1997) Exp Eye Res 64:971-978; Proia, A D. et al. (1993) Exp Eye Res 57:693-698). Briefly, neovascularization into an avascular space is monitored in vivo. This assay is commonly performed in rabbit, rat, or mouse.

The chick embryo chorioallantoic membrane assay has been used often to study tumor angiogenesis, angiogenic factors, and antiangiogenic compounds (Knighton, D. et al. (1977) Br J Cancer 35:347-356; Auerbach, R. et al. (1974) Dev Biol 41:391-394; Ausprunk, D H. et al. (1974) Dev Biol 38:237-248; Nguyen, M. et al. (1994) Microvasc Res 47:31-40). This assay uses fertilized eggs and monitors the formation of primitive blood vessels that form in the allantois, an extra-embryonic membrane.

The above is just a sampling of angiogenic inhibitor assays that may be used to assess the antiangiogenic activity of PA.

### Cancer Screening Assays:

### Mouse Models to Study Anticancer Properties of PARMs

Lewis lung carcinoma is one commonly used tumor in mice to study inhibitors of cancer. The tumor is maintained by passage from animal to animal. Mice with Lewis lung carcinomas of 600-1200 mm³ tumors are sacrificed and the skin overlying the tumor cleaned with betadine and ethanol. In a laminar flow hood, tumor tissue is excised under aseptic conditions. A suspension of tumor cells in 0.9% normal saline is made by passage of viable tumor tissue through a sieve and a series of sequentially smaller hypodermic needles of diameter 22- to 30-gauge. The final concentration is adjusted to 1×10⁷ cells/ml and the suspension is placed on ice. After the site is cleaned with ethanol, the subcutaneous dorsa of mice in the proximal midline are injected with 1×10⁶ cells in 0.1 ml of saline.

To detect inhibition with PARMs, mice can be implanted with Lewis lung carcinomas as described above. Tumors are measured with a dial-caliper and tumor volumes were determined using the formula width 2×length×0.52, and the ratio of treated to control tumor volume (T/C) was determined for the last time point. After tumor volume was 100-200 mm³ (0.5-1% of body weight), which occurs within 3-7 days, mice are randomized into two groups. One group receives a PARM injected intraperitoneal once daily. The other group receives comparable injections of the vehicle alone. The experiments are terminated and mice are sacrificed and autopsied when the control mice began to die.

The gene encoding PA (assigned Genbank accession no. M22589) has been cloned and sequenced (Ivins, B E et al., Infect. Immun. 54:537-542 (1986); Welkos, S L et al., Gene 69:287-300 (1988); U.S. Pat. No. 5,591,631, U.S. Pat. No. 5,677,274; Leppla, S H, “Anthrax Toxins,” In: Handbook of Natural Toxins: Bacterial Toxins and Virulence Factors in Disease, Moss, J. et al., eds., Dekker, New York, 1995). PA can be isolated form its natural source or it can be produced by recombinant means, or by chemical synthesis. Methods of preparing recombinant Bacillus anthracis PA or derivatives or fragments thereof have been described for use in vaccines. See, for example, U.S. patent application Ser. Nos. 6,267,966 and 6,329,156, which are herein incorporated by reference.

As described earlier, angiogenesis includes a variety of processes involving neovascularization of a tissue including “sprouting”, vasculogenesis, or vessel enlargement. With the exception of traumatic wound healing, corpus leuteum formation and embryogenesis, it is believed that the majority of angiogenesis processes are associated with disease processes and therefore the use of the present therapeutic methods are selective for the disease and do not have deleterious side effects.

There are a variety of diseases or disorders in which angiogenesis is believed to be important, referred to as angiogenic diseases including, but not limited to, obesity, inflammatory disorders such as immune and non-immune inflammation, chronic articular rheumatism and psoriasis, endometriosis, disorders associated with inappropriate or inopportune invasion of vessels such as diabetic retinopathy, macular degeneration, neovascular glaucoma, restenosis, capillary proliferation in atherosclerotic plaques and osteoporosis, and cancer associated disorders, such as solid tumors, solid tumor metastases, angiofibromas, retrolental fibroplasia, hemangiomas, Kaposi sarcoma and the like cancers which require neovascularization to support tumor growth.

As described herein, any of a variety of tissues, or organs comprised of organized tissues, can support angiogenesis in disease conditions including skin, muscle, gut, connective tissue, joints, bones and the like tissue in which blood vessels can invade upon angiogenic stimuli.

The patient treated in the present invention in its many embodiments is desirably a human patient, although it is to be understood that the principles of the invention indicate that the invention is effective with respect to all mammals, which are intended to be included in the term “patient”. In this context, a mammal is understood to include any mammalian species in which treatment of diseases associated with angiogenesis is desirable, particularly agricultural and domestic mammalian species.

Thus, in one related embodiment, a tissue to be treated is an inflamed tissue and the angiogenesis to be inhibited is inflamed tissue angiogenesis where there is neovascularization of inflamed tissue. In this class the method contemplates inhibition of angiogenesis in arthritic tissues, such as in a patient with chronic articular rheumatism, in immune or non-immune inflamed tissues, in psoriatic tissue and the like.

In another related embodiment, a tissue to be treated is a retinal tissue of a patient with a retinal disease such as diabetic retinopathy, macular degeneration or neovascular glaucoma and the angiogenesis to be inhibited is retinal tissue angiogenesis where there is neovascularization of retinal tissue.

In an additional related embodiment, a tissue to be treated is a tumor tissue of a patient with a solid tumor, metastases, a skin cancer, a breast cancer, a medullary thyroid cancer, a hemangioma or angiofibroma and the like cancer, and the angiogenesis to be inhibited is tumor tissue angiogenesis where there is neovascularization of a tumor tissue. Tumors which may be prevented or inhibited by preventing or inhibiting angiogenesis with PARMs include, but are not limited to lung tumors, pancreas tumors, breast tumors, colon tumors, laryngeal tumors, ovarian tumors, thyroid tumors, melanoma, adenocarcinoma, sarcomas, thymoma, lymphoma, liver tumors, kidney tumors, non-Hodgkins lymphoma, Hodgkins lymphoma, leukemias, uterine tumors, prostate tumors, renal tumors, brain tumors, testicular tumors, bone tumors, muscle tumors, tumors of the placenta, gastric tumors and the like.

In an additional related embodiment, a tissue to be treated is a tumor tissue of a patient with a solid tumor, metastases, a skin cancer, a breast cancer, a medullary thyroid cancer, a hemangioma or angiofibroma and the like cancer. Tumors which may be prevented or inhibited by preventing or inhibiting angiogenesis with PARM include, but are not limited to lung tumors, pancreas tumors, breast tumors, colon tumors, laryngeal tumors, ovarian tumors, thyroid tumors, melanoma, adenocarcinoma, sarcomas, thymoma, lymphoma, liver tumors, kidney tumors, non-Hodgkins lymphoma, Hodgkins lymphoma, leukemias, uterine tumors, prostate tumors, renal tumors, brain tumors, testicular tumors, bone tumors, muscle tumors, tumors of the placenta, gastric tumors and the like.

Inhibition of tumor tissue angiogenesis is a particularly preferred embodiment because of the important role neovascularization plays in tumor growth. In the absence of neovascularization of tumor tissue, the tumor tissue does not obtain the required nutrients, slows in growth, ceases additional growth, regresses and ultimately becomes necrotic resulting in killing of the tumor.

Stated in other words, the present invention provides for a method of inhibiting tumor neovascularization by inhibiting tumor angiogenesis according to the present methods. Similarly, the invention provides a method of inhibiting tumor growth by practicing the angiogenesis-inhibiting methods.

The methods are also particularly effective against the formation of metastases because (1) their formation requires vascularization of a primary tumor so that the metastatic cancer cells can exit the primary tumor and (2) their establishment in a secondary site requires neovascularization to support growth of the metastases.

In a related embodiment, the invention contemplates the practice of the method in conjunction with other therapies such as conventional chemotherapy or radiation therapy directed against solid tumors and for control of establishment of metastases. The administration of angiogenesis-inhibiting amounts of PARM may be conducted before, during or after chemotherapy or radiation therapy. In addition, the compounds of the present invention may be administered concurrently with other cancer therapies known to those of skill in the art. For example, PARM may be combined with chemotherapy, radiation, or other known angiogenesis inhibitors. Known angiogenesis inhibitors include, but are not limited to: Angiostatin, Bevacizumab (Avastin), Arresten, Canstatin, Combretastatin, Endostatin, NM-3, Thrombospondin, Tumstatin, 2-methoxyestradiol, Vitaxin, ZD1839 (Iressa), ZD6474, OS1774 (Tarceva), CI1033, PKI1666, IMC225 (Erbitux), PTK787, SU6668, SU11248, Herceptin, and IFN-α, CELEBREX® (Celecoxib), THALOMID® (Thalidomide), and IFN-α (Kerbel et al., Nature Reviews, Vol. 2, October 2002, pp. 727). For combination therapy, the dose of PARM may be administered prior to, concurrently, or after administration of a second anti-angiogenic agent or chemotherapeutic agent. Furthermore, the PARM may be administered alone or in combination with another anti-angiogenic compound prior to, concurrently, or after the surgical removal of a solid tumor mass.

In the method of treatment, the administration of PARM may be for either “prophylactic” or “therapeutic” purpose. When provided prophylactically, PARM is provided in advance of any symptom. The prophylactic administration of the PARM serves to prevent or inhibit an angiogenesis disease or disorder, i.e. cancer. Prophylactic administration of PARM may be given to a patient with, for example, a family history of cancer. Alternatively, administration of PARM may be given to a patient with rising cancer marker protein levels. Such markers include, for example, rising PSA, thymosin β-15, thymosin β-16, calcitonin, matrix metalloproteinase (MMP), and myeloma M-protein.

When provided therapeutically, PARM is provided at (or after) the onset of a symptom or indication of angiogenesis. Thus, PARM may be provided either prior to the anticipated angiogenesis at a site or after the angiogenesis has begun at a site.

Insofar as the present methods apply to inhibition of tumor neovascularization, the methods can also apply to inhibition of tumor tissue growth, to inhibition of tumor metastases formation, and to regression of established tumors.

Restenosis is a process of smooth muscle cell (SMC) migration and proliferation at the site of percutaneous transluminal coronary angioplasty which hampers the success of angioplasty. The migration and proliferation of SMC's during restenosis can be considered a process of angiogenesis which is inhibited by the present methods. Therefore, the invention also contemplates inhibition of restenosis by inhibiting angiogenesis according to the present methods in a patient following angioplasty procedures. For inhibition of restenosis, an angiogenesis-inhibiting amount of PARM is typically administered after the angioplasty. The administration of the compounds of the invention may occur from about 2 to about 28 days post-angioplasty and more typically for about the first 14 days following the procedure.

The present method for inhibiting angiogenesis in a tissue, and therefore for also practicing the methods for treatment of angiogenesis-related diseases, comprises contacting a tissue in which angiogenesis is occurring, or is at risk for occurring, with a composition comprising a therapeutically effective amount of PARM. Thus the method comprises administering to a patient a therapeutically effective amount of a physiologically tolerable composition containing PARM of the invention.

The effective dosage range for the administration of PARM depends upon the form of the PA, and its potency, as described further herein, and are amounts large enough to produce the desired effect in which angiogenesis and the disease symptoms mediated by angiogenesis are ameliorated. The dosage should not be so large as to cause adverse side effects, such as hyperviscosity syndromes, pulmonary edema, congestive heart failure, and the like. Generally, the dosage will vary with the age, condition, sex and extent of the disease in the patient and can be determined by one of skill in the art. The dosage can also be adjusted by the individual physician in the event of any complication.

A therapeutically effective amount is an amount of PARM sufficient to produce a measurable inhibition of angiogenesis or tumor growth in the tissue being treated, i.e., an angiogenesis-inhibiting amount. Inhibition of angiogenesis can be measured in situ by immunohistochemistry, or by other methods known to one skilled in the art.

One skilled in the art can readily assess the potency of a candidate PARM of this invention.

In general, it is desirable to provide the recipient with a dosage of PARM of at least about 10 μg/kg, preferably at least about 10 mg/kg or higher. A range of from about 1 μg/kg to about 100 mg/kg is preferred although a lower or higher dose may be administered. The dose provides an effective antiangiogenic serum or tissue level of PARM. The dose is administered at least once and may be provided as a bolus, a continuous administration or sustained release. Multiple administration over a period of weeks or months may be preferable. It may also be preferable to administer PARM at least once/week and even more frequent administrations (e.g. daily). Subsequent doses may be administered as indicated.

The route of administration may be intravenous (I.V.), intramuscular (I.M.), subcutaneous (S.C.), intradermal (I.D.), intraperitoneal (I.P.), intrathecal (I.T.), intrapleural, intrauterine, rectal, vaginal, topical, intratumor and the like. The compounds of the invention can be administered parenterally by injection or by gradual infusion over time and can be delivered by peristaltic means.

This invention may also be used on a stent or other medical device to prevent angiogenesis and restenosis in the tissue in which it is implanted.

Administration may be by transmucosal or transdermal means. For transmucosal or transdermal administration, penetrants appropriate to the barrier to be permeated are used in the formulation. Such penetrants are generally known in the art, and include, for example, for transmucosal administration bile salts and fusidic acid derivatives. In addition, detergents may be used to facilitate permeation. Transmucosal administration may be through nasal sprays, for example, or using suppositories. For oral administration, the compounds of the invention are formulated into conventional oral administration forms such as capsules, tablets and tonics.

For topical administration, PARM is formulated into ointments, salves, gels, or creams, as is generally known in the art.

The therapeutic compositions of this invention are conventionally administered intravenously, as by injection of a unit dose, for example. The term “unit dose” when used in reference to a therapeutic composition of the present invention refers to physically discrete units suitable as unitary dosage for the subject, each unit containing a predetermined quantity of active material calculated to produce the desired therapeutic effect in association with the required diluent; i.e., carrier, or vehicle.

The compositions are administered in a manner compatible with the dosage formulation, and in a therapeutically effective amount. The quantity to be administered and timing depends on the subject to be treated, capacity of the subject's system to utilize the active ingredient, and degree of therapeutic effect desired. Precise amounts of active ingredient required to be administered depend on the judgment of the practitioner and are peculiar to each individual.

### Therapeutic Compositions

The PARMs useful for practicing the methods of the present invention are described herein. Any formulation or drug delivery system containing the active ingredients, which is suitable for the intended use, as are generally known to those of skill in the art, can be used. Suitable pharmaceutically acceptable carriers for oral, rectal, topical or parenteral (including inhaled, subcutaneous, intraperitoneal, intramuscular and intravenous) administration are known to those of skill in the art. The carrier must be pharmaceutically acceptable in the sense of being compatible with the other ingredients of the formulation and not deleterious to the recipient thereof.

As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects.

Formulations suitable for parenteral administration conveniently include sterile aqueous preparation of the active compound which is preferably isotonic with the blood of the recipient. Thus, such formulations may conveniently contain distilled water, 5% dextrose in distilled water or saline. Useful formulations also include concentrated solutions or solids containing the compound which upon dilution with an appropriate solvent give a solution suitable for parental administration above.

For enteral administration, a compound can be incorporated into an inert carrier in discrete units such as capsules, cachets, tablets or lozenges, each containing a predetermined amount of the active compound; as a powder or granules; or a suspension or solution in an aqueous liquid or non-aqueous liquid, e.g., a syrup, an elixir, an emulsion or a draught. Suitable carriers may be starches or sugars and include lubricants, flavorings, binders, and other materials of the same nature.

A tablet may be made by compression or molding, optionally with one or more accessory ingredients. Compressed tablets may be prepared by compressing in a suitable machine the active compound in a free-flowing form, e.g., a powder or granules, optionally mixed with accessory ingredients, e.g., binders, lubricants, inert diluents, surface active or dispersing agents. Molded tablets may be made by molding in a suitable machine, a mixture of the powdered active compound with any suitable carrier.

A syrup or suspension may be made by adding the active compound to a concentrated, aqueous solution of a sugar, e.g., sucrose, to which may also be added any accessory ingredients. Such accessory ingredients may include flavoring, an agent to retard crystallization of the sugar or an agent to increase the solubility of any other ingredient, e.g., as a polyhydric alcohol, for example, glycerol or sorbitol.

Formulations for rectal administration may be presented as a suppository with a conventional carrier, e.g., cocoa butter or Witepsol S55 (trademark of Dynamite Nobel Chemical, Germany), for a suppository base.

Formulations for oral administration may be presented with an enhancer. Orally-acceptable absorption enhancers include surfactants such as sodium lauryl sulfate, palmitoyl carnitine, Laureth-9, phosphatidylcholine, cyclodextrin and derivatives thereof; bile salts such as sodium deoxycholate, sodium taurocholate, sodium glycochlate, and sodium fusidate; chelating agents including EDTA, citric acid and salicylates; and fatty acids (e.g., oleic acid, lauric acid, acylcarnitines, mono- and diglycerides). Other oral absorption enhancers include benzalkonium chloride, benzethonium chloride, CHAPS (3-(3-cholamidopropyl)-dimethylammonio-1-propanesulfonate), Big-CHAPS(N,N-bis(3-D-gluconamidopropyl)-cholamide), chlorobutanol, octoxynol-9, benzyl alcohol, phenols, cresols, and alkyl alcohols. An especially preferred oral absorption enhancer for the present invention is sodium lauryl sulfate.

Alternatively, the compound may be administered in liposomes or microspheres (or microparticles). Methods for preparing liposomes and microspheres for administration to a patient are well known to those of skill in the art. U.S. Pat. No. 4,789,734, the contents of which are hereby incorporated by reference, describes methods for encapsulating biological materials in liposomes. A review of known methods is provided by G. Gregoriadis, Chapter 14, “Liposomes,” Drug Carriers in Biology and Medicine, pp. 287-341 (Academic Press, 1979).

Microspheres formed of polymers or proteins are well known to those skilled in the art, and can be tailored for passage through the gastrointestinal tract directly into the blood stream. Alternatively, the compound can be incorporated and the microspheres, or composite of microspheres, implanted for slow release over a period of time ranging from days to months. See, for example, U.S. Pat. Nos. 4,906,474, 4,925,673 and 3,625,214, and Jein, TIPS 19:155-157 (1998), the contents of which are hereby incorporated by reference.

In one embodiment, PARM can be formulated into a liposome or microparticle which is suitably sized to lodge in capillary beds following intravenous administration. When the liposome or microparticle is lodged in the capillary beds surrounding ischemic tissue, the agents can be administered locally to the site at which they can be most effective. Suitable liposomes for targeting ischemic tissue are generally less than about 200 nanometers and are also typically unilamellar vesicles, as disclosed, for example, in U.S. Pat. No. 5,593,688 to Baldeschweiler, entitled “Liposomal targeting of ischemic tissue,” the contents of which are hereby incorporated by reference.

Preferred microparticles are those prepared from biodegradable polymers, such as polyglycolide, polylactide and copolymers thereof. Those of skill in the art can readily determine an appropriate carrier system depending on various factors, including the desired rate of drug release and the desired dosage.

In one embodiment, the formulations are administered via catheter directly to the inside of blood vessels. The administration can occur, for example, through holes in the catheter. In those embodiments wherein the active compounds have a relatively long half life (on the order of 1 day to a week or more), the formulations can be included in biodegradable polymeric hydrogels, such as those disclosed in U.S. Pat. No. 5,410,016 to Hubbell et al. These polymeric hydrogels can be delivered to the inside of a tissue lumen and the active compounds released over time as the polymer degrades. If desirable, the polymeric hydrogels can have microparticles or liposomes which include the active compound dispersed therein, providing another mechanism for the controlled release of the active compounds.

The formulations may conveniently be presented in unit dosage form and may be prepared by any of the methods well known in the art of pharmacy. All methods include the step of bringing the active compound into association with a carrier which constitutes one or more accessory ingredients. In general, the formulations are prepared by uniformly and intimately bringing the active compound into association with a liquid carrier or a finely divided solid carrier and then, if necessary, shaping the product into desired unit dosage form.

The formulations may further include one or more optional accessory ingredient(s) utilized in the art of pharmaceutical formulations, e.g., diluents, buffers, flavoring agents, binders, surface active agents, thickeners, lubricants, suspending agents, preservatives (including antioxidants) and the like.

PARM may be presented for administration to the respiratory tract as a snuff or an aerosol or solution for a nebulizer, or as a microfine powder for insufflation, alone or in combination with an inert carrier such as lactose. In such a case the particles of active compound suitably have diameters of less than 50 microns, preferably less than 10 microns, more preferably between 2 and 5 microns.

A formulation for the administration of protein via the nasal route is described in U.S. Pat. No. 5,759,565, and can be modified for PARM. This nasal formulation is designed to be stored in a multi-dose container, is stable for an extended period of time, and resists bacterial contamination. The preservative in the formulation, benzalkonium chloride, enhances the absorption of the protein.

Generally for nasal administration a mildly acid pH will be preferred. Preferably the compositions of the invention have a pH of from about 3 to 5, more preferably from about 3.5 to about 3.9 and most preferably 3.7. Adjustment of the pH is achieved by addition of an appropriate acid, such as hydrochloric acid.

The preparation of a pharmacological composition that contains active ingredients dissolved or dispersed therein is well understood in the art and need not be limited based on formulation. Typically such compositions are prepared as injectables either as liquid solutions or suspensions, however, solid forms suitable for solution, or suspensions, in liquid prior to use can also be prepared. The preparation can also be emulsified.

The active ingredient can be mixed with excipients which are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient.

PARM of the present invention can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like.

Physiologically tolerable carriers are well known in the art. Exemplary of liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes.

Liquid compositions can also contain liquid phases in addition to and to the exclusion of water. Exemplary of such additional liquid phases are glycerin, vegetable oils such as cottonseed oil, and water-oil emulsions.

A therapeutic composition contains an angiogenesis-inhibiting amount of PARM of the present invention.

### Polypeptides

A polypeptide (peptide) PARM can have the sequence characteristics of either natural PA or a fragment, analog, or derivative of PA. Full length, human PA peptide contains the amino acid sequence set forth in SEQ ID. NO 1. PA is an 83 kDa protein with 4 domains. It is secreted in a soluble form which can then bind to cell surface receptors which include ATR1/TEM8 (anthrax toxin receptor 1/tumor endothelial marker 8) and ATR2/CMG2 (anthrax toxin receptor 2/capillary morphogenesis gene 2). Once at the cell surface, PA is cleaved by furin-type proteases resulting in 63 and 20 kDa polypeptides (PA63 and PA20) that can remain associated with each other. Once cleaved, PA63 can oligomerize into a heptamer. When the 20 kDa polypeptide fragments release, seven A-subunit binding sites are revealed, of which up to three can be occupied by any combination of A-subunits. When this complex is endocytosed and exposed to low pH, the PA heptamer rearranges to form a transmembrane pore, which then introduces the A-subunits into the cell (Collier, R. J., and Young, J. A. Annu Rev Cell Dev Biol 19, 45-70, 2003). None of the A-subunits is toxic or active without PA and PA has no known natural function without the A-subunits.

It should be understood that a subject the polypeptide PARM need not be identical to the amino acid sequence of human PA (SEQ ID. NO 1), so long as it has 50% identity to the derivative of PA from which it was derived and has angiogenesis inhibiting activity. In another embodiment, the derivative of PA has at least 75% identity to the PA derivative from which it was derived. In a most preferred embodiment, the derivative of PA has at least 90% identity to the PA from which it was derived. In one preferred embodiment, PA has a mutation that prevents furin cleavage, such as described in Klimpel, K R, et al. Proc. Natl. Acad. Sci. USA 89(21):10277-10281, 1992.

A subject PARM includes any analog, fragment or chemical derivative of a polypeptide whose amino acid residue sequence is shown herein so long as the polypeptide is angiogenesis-inhibiting or cancer-inhibiting. Therefore, a present polypeptide can be subject to various changes, substitutions, insertions, and deletions where such changes provide for certain advantages in its use. In this regard, the PARM of this invention corresponds to, rather than is identical to, the sequence of a recited peptide where one or more changes are made and it retains the ability to function as an angiogenesis inhibitor in one or more of the assays as defined herein.

Thus, a PARM can be in any of a variety of forms of peptide derivatives, which include amides, conjugates with proteins, cyclic peptides, polymerized peptides, analogs, fragments, chemically modified peptides, and the like derivatives.

The term “analog” includes any polypeptide having an amino acid residue sequence substantially identical to a sequence specifically shown herein in which one or more residues have been conservatively substituted with a functionally similar residue and which displays angiogenesis-inhibiting activity as described herein. Examples of conservative substitutions include the substitution of one non-polar (hydrophobic) residue such as isoleucine, valine, leucine or methionine for another, the substitution of one polar (hydrophilic) residue for another such as between arginine and lysine, between glutamine and asparagine, between glycine and serine, the substitution of one basic residue such as lysine, arginine or histidine for another, or the substitution of one acidic residue, such as aspartic acid or glutamic acid for another.

The phrase “conservative substitution” also includes the use of a chemically derivatized residue in place of a non-derivatized residue provided that such polypeptide displays the requisite inhibition activity.

A “chemical derivative” refers to a subject polypeptide having one or more residues chemically derivatized by reaction of a functional side group. In addition to side group derivitations, a chemical derivative can have one or more backbone modifications including alpha-amino substitutions such as N-methyl, N-ethyl, N-propyl and the like, and alpha-carbonyl substitutions such as thioester, thioamide, guanidino and the like. Such derivatized molecules include for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine. Also included as chemical derivatives are those peptides which contain one or more naturally occurring amino acid derivatives of the twenty standard amino acids. Also included as chemical derivatives are those peptides which contain one or more commonly available, non-natural amino acids. For example those available for peptide synthesis from commercial suppliers (e.g. Bachem Catalog, 2004 pp. 1-276). For examples: 4-hydroxyproline may be substituted for proline; 5-hydroxylysine may be substituted for lysine; 3-methylhistidine may be substituted for histidine; homoserine may be substituted for serine; ornithine may be substituted for lysine; β-alanine may be substituted for alanine; norleucine may be substituted for leucine; phenylglycine may be substituted for phenylalanine, and L-1,2,3,4-tetrahydronorharman-3-carboxylic acid or H-β-(3-Benzothienyl)-Ala-OH may be substituted for tryptophan. Polypeptides of the present invention also include any polypeptide having one or more additions and/or deletions or residues relative to the sequence of a polypeptide whose sequence is shown herein, so long as the requisite activity is maintained.

As with all therapies involving proteins and peptides, reducing immunogenicity and prolonging half-life may be necessary to enhance the efficacy (Hermeling Pharm Res. 2004 June; 21(6):897-903.). Such methods to reduce immunogenicity are numerous including such well known examples as the conjugation of the protein with polyalkylene glycols (such as polyethylene glycol/PEG and polyethylene oxide), the alteration of amino acids to reduce potential T cell epitopes, co-administration with immunosuppressive drugs and production of fusion proteins (such as Fc antibody fragments fusion proteins).

The term “fragment” refers to any subject polypeptide having an amino acid residue sequence shorter than that of a polypeptide whose amino acid residue sequence is shown herein.

When a polypeptide of the present invention has a sequence that is not identical to the sequence of PA, it is typically because one or more conservative or non-conservative substitutions have been made, usually no more than about 30 number percent, and preferably no more than 10 number percent of the amino acid residues are substituted. Additional residues may also be added at either terminus of a polypeptide for the purpose of providing a “linker” by which the polypeptides of this invention can be conveniently affixed to a label or solid matrix, or carrier.

Labels, solid matrices and carriers that can be used with the polypeptides of this invention are described herein.

Amino acid residue linkers are usually at least one residue and can be 40 or more residues, more often 1 to 10 residues and may couple polypeptides or proteins covalently or non-covalently. Typical amino acid residues used for linking are glycine, tyrosine, cysteine, lysine, glutamic and aspartic acid, or the like. In addition, a subject polypeptide can differ, unless otherwise specified, from the natural sequence of PA by the sequence being modified by terminal-NH₂ acylation, e.g., acetylation, or thioglycolic acid amidation, by terminal-carboxylamidation, e.g., with ammonia, methylamine, and the like terminal modifications. Terminal modifications are useful, as is well known, to reduce susceptibility by proteinase digestion, and therefore serve to prolong half life of the polypeptides in solutions, particularly biological fluids where proteases may be present.

Peptide sequences of the present invention may also be linked together using non-peptide crosslinkers (Pierce 2003-2004 Applications Handbook and Catalog, Chapter 6) or other scaffolds such as HPMA, polysextran, polysacchrides, ethylene-glycol, poly-ethylene-glycol, glycerol, sugars, and sugar alchohols (e.g. sorbitol, mannitol). Such linked peptide may be composed of one or more, identical or different sequences or subsequences of PARM. For example, a peptide comprising a portion of domain 2 of SEQ ID NO 1, such as amino acids 365-384 of sequence SEQ ID NO 1, may be coupled to the amine-reactive moity of a heterobifunctional crosslinker such as AMAS (N-[α-Maleimidoacetoxy]succinimide ester) or Sulfo-SMPB (Sulfosuccinimidyl 4-[p-maleimidophenly]-butyrate). This may also be coupled to domain 4 of SEQ ID NO 1 in which a single amino-acid has been changed to a cysteine or to domain 4 subsequences such as amino acids 708-721 or 676-721 of SEQ ID NO 1 in which a single amino-acid has been changed to a cysteine. This will result in linked peptides comprising a portion of domains 2 and 4 of SEQ ID NO 1.

Any peptide of the present invention may be used in the form of a pharmaceutically acceptable salt. Suitable acids which are capable of forming salts with the peptides of the present invention include inorganic acids such as trifluoroacetic acid (TFA), trichloroacetic acid (TCA), hydrochloric acid (HCl), hydrobromic acid, perchloric acid, nitric acid, thiocyanic acid, sulfuric acid, methane sulfonic acid, acetic acid, phosphoric acetic acid, propionic acid, glycolic acid, lactic acid, pyruvic acid, oxalic acid, malonic acid, succinic acid, maleic acid, fumaric acid, anthranilic acid, cinnamic acid, naphthalene sulfonic acid, sulfanilic acid or the like. HCl and TFA salts are particularly preferred.

Suitable bases capable of forming salts with the peptides of the present invention include inorganic bases such as sodium hydroxide, ammonium hydroxide, potassium hydroxide and the like; and organic bases such as mono-, di- and tri-alkyl and aryl amines (e.g. triethylamine, diisopropyl amine, methyl amine, dimethyl amine and the like) and optionally substituted ethanolamines (e.g. ethanolamine, diethanolamine and the like).

A peptide of the present invention also referred to herein as a subject polypeptide, can be synthesized by any of the techniques that are known to those skilled in the polypeptide art, including recombinant DNA techniques. Synthetic chemistry techniques, such as a solid-phase Merrifield-type synthesis, are preferred for reasons of purity, antigenic specificity, freedom from undesired side products, ease of production and the like. An excellent summary of the many techniques available can be found in Steward et al., “Solid Phase Peptide Synthesis”, W. H. Freeman Co., San Francisco, 1969; Bodanszky, et al., “Peptide Synthesis”, John Wiley & Sons, Second Edition, 1976; J. Meienhofer, “Hormonal Proteins and Peptides”, Vol. 2, p. 46, Academic Press (New York), 1983; Merrifield, Adv. Enzymol., 32:221-96, 1969; Fields et al., Int. J. Peptide Protein Res., 35:161-214, 1990; and U.S. Pat. No. 4,244,946 for solid phase peptide synthesis, and Schroder et al., “The Peptides”, Vol. 1, Academic Press (New York), 1965 for classical solution synthesis, each of which is incorporated herein by reference. Appropriate protective groups usable in such synthesis are described in the above texts and in J. F. W. McOmie, “Protective Groups in Organic Chemistry”, Plenum Press, New York, 1973, which is incorporated herein by reference.

In general, the solid-phase synthesis methods contemplated comprise the sequential addition of one or more amino acid residues or suitably protected amino acid residues to a growing peptide chain. Normally, either the amino or carboxyl group of the first amino acid residue is protected by a suitable, selectively removable protecting group. A different, selectively removable protecting group is utilized for amino acids containing a reactive side group such as lysine.

Using a solid phase synthesis as exemplary, the protected or derivatized amino acid is attached to an inert solid support through its unprotected carboxyl or amino group. The protecting group of the amino or carboxyl group is then selectively removed and the next amino acid in the sequence having the complimentary (amino or carboxyl) group suitably protected is admixed and reacted under conditions suitable for forming the amide linkage with the residue already attached to the solid support. The protecting group of the amino or carboxyl group is then removed from this newly added amino acid residue, and the next amino acid (suitably protected) is then added, and so forth. After all the desired amino acids have been linked in the proper sequence, any remaining terminal and side group protecting groups (and solid support) are removed sequentially or concurrently, to afford the final linear polypeptide.

The resultant linear polypeptides prepared for example as described above may be reacted to form their corresponding cyclic peptides. An exemplary method for preparing a cyclic peptide is described by Zimmer et al., Peptides 1992, pp. 393-394, ESCOM Science Publishers, B.V., 1993. Typically, tertbutoxycarbonyl protected peptide methyl ester is dissolved in methanol and sodium hydroxide solution are added and the admixture is reacted at 20° C. to hydrolytically remove the methyl ester protecting group. After evaporating the solvent, the tertbutoxycarbonyl protected peptide is extracted with ethyl acetate from acidified aqueous solvent. The tertbutoxycarbonyl protecting group is then removed under mildly acidic conditions in dioxane cosolvent. The unprotected linear peptide with free amino and carboxy termini so obtained is converted to its corresponding cyclic peptide by reacting a dilute solution of the linear peptide, in a mixture of dichloromethane and dimethylformamide, with dicyclohexylcarbodiimide in the presence of 1-hydroxybenzotriazole and N-methylmorpholine. The resultant cyclic peptide is then purified by chromatography.

Alternative methods for cyclic peptide synthesis are described by Gurrath et al., Eur. J. Biochem., 210:911-921 (1992).

In addition, PARM can be provided in the form of a fusion protein. Fusion proteins are proteins produced by recombinant DNA methods as described herein in which the subject polypeptide is expressed as a fusion with a second carrier protein such as a glutathione sulfhydryl transferase (GST) or other well known carrier.

In one preferred embodiment, PA fragments, analogs, or derivatives thereof are linked together via a peptide or other linker. A “peptide linker” is a short (e.g., about 1-40, e.g., 1-20 amino acids) sequence of amino acids that is not part of the sequence of either of two polypeptides being joined. A linker peptide is attached on its amino-terminal end to one polypeptide or polypeptide domain and on its carboxyl-terminal end to another polypeptide or polypeptide domain. Examples of useful linker peptides include, but are not limited to, glycine polymers ((G)n) including glycine-serine and glycine-alanine polymers (e.g., a (Gly4Ser)n repeat where n=1-8, preferably, n=3, 4, 5, or 6). PA fragments, analogs, or derivatives thereof can also be joined by chemical bond linkages, such as linkages by disulfide bonds or by chemical bridges.

### Gene Therapy

The PARM of the present invention may be administered to a patient by any one of several gene therapy techniques known to those of skill in the art. In general, gene therapy can be accomplished by either direct transformation of target cells within the mammalian subject (in vivo gene therapy) or transformation of cells in vitro and subsequent implantation of the transformed cells into the mammalian subject (ex vivo gene therapy).

U.S. Pat. No. 6,531,456 provides methods for the successful transfer of a gene into a solid tumor cell using recombinant AAV virions. Generally, the method described in U.S. Pat. No. 6,531,456 allows for the direct, in vivo injection of recombinant AAV virions into tumor cell masses, e.g., by intra-tumoral injection. The invention also provides for the simultaneous delivery of a second gene using the recombinant AAV virions, wherein the second gene is capable of providing an ancillary therapeutic effect when expressed within the transduced cell.

The recombinant AAV virions described above, including the DNA of interest, can be produced using standard methodology, known to those of skill in the art. The methods generally involve the steps of (1) introducing an AAV vector into a host cell; (2) introducing an AAV helper construct into the host cell, where the helper construct includes AAV coding regions capable of being expressed in the host cell to complement AAV helper functions missing from the AAV vector; (3) introducing one or more helper viruses and/or accessory function vectors into the host cell, wherein the helper virus and/or accessory function vectors provide accessory functions capable of supporting efficient recombinant AAV (“rAAV”) virion production in the host cell; and (4) culturing the host cell to produce rAAV virions. The AAV vector, AAV helper construct and the helper virus or accessory function vector(s) can be introduced into the host cell either simultaneously or serially, using standard transfection techniques.

PARMs used in the methods of the present invention can be delivered systemically via in vivo gene therapy. Systemic treatment involves transfecting target cells with the DNA of interest, i.e. PA or PA fragments, analogs, or derivatives thereof, expressing the coded protein in that cell, and the capability of the transformed cell to subsequently secrete the manufactured protein into blood.

A variety of methods have been developed to accomplish in vivo transformation including mechanical means (e.g, direct injection of nucleic acid into target cells or particle bombardment), recombinant viruses, liposomes, and receptor-mediated endocytosis (RME) (for reviews, see Chang et al. 1994 Gastroenterol. 106:1076-84; Morsy et al. 1993 JAMA 270:2338-45; and Ledley 1992 J. Pediatr. Gastroenterol. Nutr. 14:328-37).

### EXAMPLES

### I. Anthrax Protective Antigen (PA) Inhibits Angiogenesis

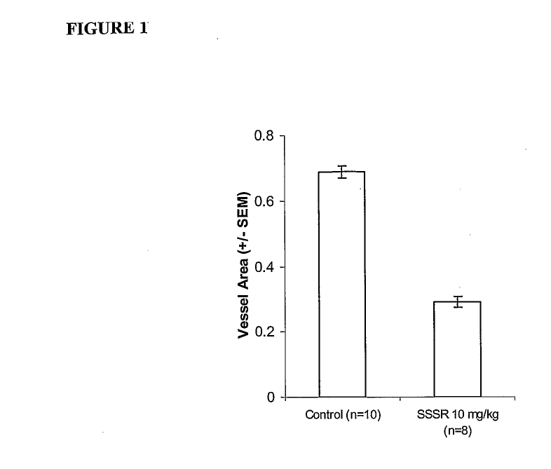

We hypothesized that protective antigen (PA) could inhibit angiogenesis by binding to endothelial cell-surface receptors including CMG2 or TEM8. In a standard corneal neovascularization assay, we tested a mutant version of PA (SSSR) that can not be cleaved by furin protease to form PA oligomers (which form pores in cells and can be internalized). Protein (at 10 mg/kg) was injected daily for 6 days and the eyes were read. As shown in FIG. 1, SSSR inhibited VEGF-induced angiogenesis by about 60%.

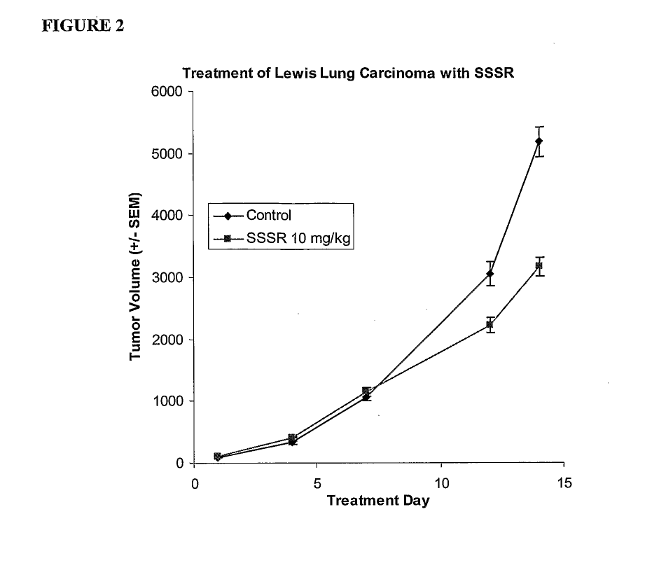

We then tested the ability of this protein to inhibit tumor growth. Lewis-lung carcinoma tumors were implanted in the backs of C57BL/6J mice. Once the tumor reached a size of 100 mm³, the mice were treated daily at 10 mg/kg with SSSR. The results are shown in FIG. 2. SSSR inhibited tumor growth by about 40%.

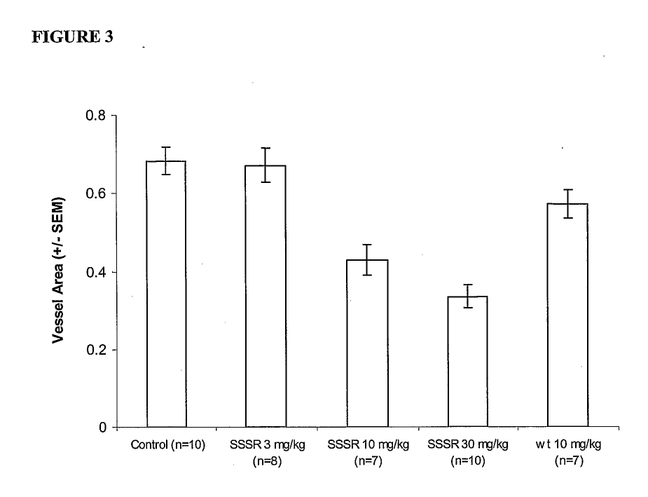

We compared the effects of SSSR and wild-type PA which will be cleaved in-situ to at least two fragments, one ˜20 kDa, and one ˜63 kDa, on VEGF-induced angiogenesis in a standard corneal neovascularization assay. FIG. 3 demonstrates that while both have activity, SSSR has a greater specific activity.

1. A method of inhibiting cancer or angiogenesis in a tissue of a mammal having an angiogenic disease/disorder; or at risk for developing cancer or an angiogenic disease/disorder comprising administering to said mammal a pharmaceutical composition comprising a cancer inhibiting or an angiogenesis-inhibiting amount of a PARM, wherein the composition is substantially free of anthrax lethal factor or other toxins.

2. The method of claim 1, wherein PARM comprises amino acids 1-764 of SEQ ID NO.: 1.

3. The method of claim 1, wherein PARM comprises amino acids 30-764 of SEQ ID NO.: 1.

4. The method of claim 1, wherein PARM comprises amino acids 365-384 of SEQ ID NO.: 1.

5. The method of claim 1, wherein PARM comprises amino acids 708-721 of SEQ ID NO.: 1.

6. The method of claim 1, wherein PARM comprises amino acids 676-694 of SEQ ID NO.: 1.

7. The method of claim 1, wherein PARM comprises amino acids 732-751 of SEQ ID NO.: 1.

8. The method of claim 1, wherein PARM comprises amino acids 595-764 of SEQ ID NO.: 1.

9. The method of claim 1, wherein PARM comprises a PA derivative having at least 50% identity compared to a fragment of PA from which the derivative was derived, wherein the derivative is derived from SEQ ID NO.1.

10. The method of claim 9, wherein said PARM further comprises a conjugated protein, or a cyclic peptide, or a polymerized peptide, or a chemically modified peptide, or linked peptides, or combinations thereof, and like derivatives.

11. The method of claim 1, wherein PARM is a mutant PA that can not be cleaved by furin.

12. The method of claim 1 wherein PARM is native PA.

13. The method of claim 1 wherein cancer is treated with an PARM.

14. The method of claim 1, wherein said angiogenic disease is retinopathy of prematurity, diabetic retinopathy or macular degeneration.

15. The method of claim 1, wherein said mammal is at risk for atherosclerosis.

16. The method of claim 1, wherein said disease or disorder is arthritis or rheumatoid arthritis.

17. The method of claim 1, wherein said administering is conducted in conjunction with chemotherapy.

18. The method of claim 1, wherein said administering is conducted in conjunction with radiation therapy.

19. The method of claim 1, wherein said administering is conducted in conjunction with other known angiogenesis inhibitors.

20. The method of claim 1, wherein said administering comprises intravenous, intramuscular, subcutaneous, intradermal, topical, intraperitoneal, intrathecal, intrapleural, intrauterine, rectal, vaginal, intrasynovial, intraocular/periocular, intratumor or parenternal administration.

21. The method of claim 1, wherein said administering comprises a gene therapy vector that constitutively expresses or inducibly expresses said PARM.

22. The method of claim 1, wherein said PARM is administered prophylactically.

23. The method of claim 1, wherein said PARM is administered therapeutically.

24. The method of claim 1, wherein said mammal is at risk for developing said angiogenic disease or disorder.

25. The method of claim 1, wherein said PARM is incorporated into a stent for local release and inhibition of restenosis.

26. The method of claim 24, wherein said risk for developing an angiogenic disease or disorder is determined genetically.

27. The method of claim 24, wherein said risk for developing an angiogenic disease or disorder is determined by measuring levels of cancer marker protein.

28. The method of claim 27, wherein the cancer marker protein is selected from the group consisting of calcitonin, PSA, thymosin β-15, thymosin β-16, or matrix metalloproteinase (MMP).
